(19) United States
(12) Patent Application Publication
(10) Pub. No.: US 2008/0139435 A1
(43) Pub. Date: Jun. 12, 2008

(54) Sanitizing Method

(75) Inventor: Keith M. MacGregor, West Yorkshire (GB)

Correspondence Address: CONNOLLY BOVE LODGE & HUTZ LLP, 1875 EYE STREET, N.W., SUITE 1100, WASHINGTON, DC, 20036, US

(73) Assignee: Ebiox, Ltd., Warrington (GB)

(21) Appl. No.: 11/571,144

(22) PCT Filed: Jun. 20, 2005
(86) PCT No.: PCT/GB05/02432

§ 371 Date: Nov. 15, 2007

(30) Foreign Application Priority Data
Jun. 22, 2004 (GB) 0413950.7

(51) Int. Cl. C11D 3/04

(52) U.S. Cl. 510/161

(57) ABSTRACT

Ductwork used for the supply of potable water in dental surgeries is prone to undesirable build up of biofilm and associated bacteria. These may be combated effectively by applying to the ductwork a composition comprising: a metal silicate or silicate complex; a metal phosphate or polyphosphate or orthophosphoric acid; and a non-ionic or amphoteric surfactant.

The present invention concerns a method for sanitizing ductwork and fittings associated with the supply of potable liquids. The invention relates particularly, but not exclusively, to a method of sanitizing ductwork and fittings associated with water supply in a dental facility.

It has been recognized that there is a problem with the hygiene of the supply of water (under which term we include water-based mouth swills and the like) in dental facilities. Dental facilities often have small bore systems to deliver potable water to patients in the dental chair. Often the water is gravity fed from containers—often as small as 1 litre—which are topped up as required.

It has been recognized that certain dental apparatus may permit the spread and dissemination of pathogens, typically bacteria. Their growth and dissemination is assisted by biofilms.

Biofilms form when micro-organisms adhere to a surface. They grow and become a culture medium for more micro-organisms. A biofilm can be formed by a single species or micro-organism, for example a bacterium, fungus, algae, or protozoa. However, biofilms may often be formed by multiple species of micro-organism; for example they may often be formed of multiple species of bacteria. Alternatively or additionally they may be formed from debris. The debris may be from living organisms, for example sebum or dead skin cells. Alternatively or additionally the debris may be from inanimate sources, for example corrosion products.

The formation of biofilms is a serious problem with potentially grave consequences in dental facilities where, harmful pathogens such as Legionella (responsible for legionnaires disease) may be present. The need to develop technologies to stop these bacteria from spreading is paramount.

There have been proposals to sanitize the ductwork associated with the supply of water in dental facilities but to the best of our knowledge these have not been successful. Our assessment of the reasons is as follows. The focus has been on combating the pathogens. This appears to be the logical approach; it is the pathogens that damage patients. One such example is given in US Patent Application No. US2002/0195406 (Kross, Wade, published 26 Dec. 2002). In this document there is described the combating of microbial flora associated with biofilm by exposing it to chlorine dioxide, to reduce or eliminate such microbial flora. The inventors assert that the chlorine dioxide “has the apparent ability to penetrate biofilms with a much greater efficiency and destroy deep-lying bacteria”.

A similar approach is reflected in scientific publications. It was noted in New Scientist (2 Sep. 2000) that the American Dental Association (ADA) that surgeries “should use bottled water and disinfectants to reduce the risk from bacteria”. Smith et al (British Dental Journal, Vol. 186, No. 1, 9 Jan. 1999) discuss the problem and the difficulty in using contamination strategies to combat e.g. Legionella species and coliforms such as E. Coli.

There is thus emphasis in the art on the micro-organisms. In our view this approach, although apparently the logical one, is doomed to failure. This is because the biofilm remains in place, ready to serve as a substrate for new populations of pathogens, or for residual colonies of pathogens which have not been destroyed. Some have simply not given a high enough level of kill of target pathogens; or a high enough degradation level of the biofilm.

We believe that if a sanitizing composition leaves the biofilm in a viable state, effective sanitizing treatment over a useful period is not achievable. In that circumstance all that happens is that the pathogen/biofilm axis is given an intermittent setback. The pathogens simply regrow.

Further, we believe to that to damage the biofilm but not to destroy it may actually be detrimental to dental patients. This is because a damaged biofilm may be release pathogens more readily that an intact biofilm will. Furthermore the patient is more likely to encounter detached flakes of biofilm from their water supply during dental procedures. This may be particularly unhygienic as well as being unpleasant, and even disturbing, for the patients.

It follows that in our analysis the correct route to effective sanitization is to attack the biofilm which supports the pathogens; and that the attack on the biofilm needs to be highly effective, or even “all or nothing”.

If the biofilm is completely removed and is prevented from re-forming there is nowhere for the pathogens to colonise.

It is an object of embodiments of the present invention to provide an improved method for sanitizing ductwork and fittings associated with the supply of potable liquids, especially potable water in dental facilities, by providing effective combating of biofilm.

According to the present invention there is provided a method of sanitizing ductwork for supply of a potable liquid, the method comprising applying to the ductwork an aqueous composition comprising:
a metal silicate or silicate complex;
a metal phosphate or polyphosphate;
or orthophosphoric acid; and
a non-ionic or amphoteric surfactant.

The composition optionally includes one or more of:
an oxygen donor;
an activator for the oxygen donor;
a sequestering agent;
a quaternary ammonium salt;
an alcohol;
an acidifier;
a corrosion inhibitor;
a base, for example sodium carbonate;
a cationic surfactant or source of cations; and
colours, fragrances, and/or anti-foaming agents as required.

In the following definitions, and throughout this specification, reference to X% of a component denotes X% by weight of that component, on total weight of the composition (with reference to the composition that is actually used in the method of the present invention).

Preferably, the metal cation in the silicate or silicate complex is an alkali metal cation or an ammonium cation. More preferably, the cation is sodium.

Preferably the composition comprises 0.001 to 5% of the silicate or silicate complex present, more preferably 0.01 to 0.5%, and most preferably 0.03 to 0.2%. The silicate complex may be in the form of a metasilicate, ortho-silicate, thiosilicate, pyrosilicate and other similar complex silicates. Metasilicates are particularly preferred.

Ideally, the metal phosphate or polyphosphate is an alkali metal or ammonium phosphate and the phosphate may be mono, di, or tribasic. Tetrasodium pyrophosphate is the most preferred phosphate. In the case of a liquid formulation, there is preferably 0.001 to 10% of the phosphate or polyphosphate or orthophosphoric acid present; more preferably 0.05 to 5%, especially 0.2 to 2%.

Any conventional non-ionic surfactant may be used in the compositions of the present invention. However, the surfactant of choice is NP-9 (nonyl phenol ethoxylate) which is available from ICI under the trade name Synperonic N.

Preferably there is 0.001 to 2% of the non-ionic surfactant present, more preferably 0.005 to 1%, especially 0.01 to 0.5%.

Amphoteric surfactants may also be used in the compositions of the present invention in the same amounts as for non-ionic surfactants. The type of non-ionic or amphoteric surfactant and the chain length will depend on the reactivity needs of the formulation and may readily be determined by experimentation. It is, however, preferred for the isoelectric point to be higher than pH7. Carboxylated imidazolenes are the preferred amphoteric surfactants.

The oxygen donor, when present, is preferably a peroxygen compound. Preferably it generates peracetic acid in situ. By manipulating the components and concentrations the rate of peracetic acid release can be tailored to the ducting disinfection requirement. The oxygen donor, when present, further serves to loosen materials from surfaces being cleaned. Preferably the oxygen donor is sodium perborate monohydrate. The oxygen donor is preferably present in an amount of 0.001 to 5%, more preferably 0.01 to 2%, especially from 0.1 to 1%. The oxygen donor has the effect of significantly improving the cleaning on non-metallic materials such as plastics ducting. Other suitable oxygen donors include percarbamate, manganese salts, aluminium alkoxides and oxyhalides.

The activator for the oxygen donor is preferably a peroxide activator, able to deliver broad spectrum anti-microbial activity. Preferred is TAED (tetraacetyl ethylene diamine). An activator is preferably present in an amount from 0.001 to 2%, more preferably 0.01 to 1%, most preferably 0.05 to 0.5%.

The quaternary ammonium salt which may be present acts as a complexing agent and may be derived from a lower alkyl amine having one or more alkyl substituents each being C6 or less and a single substituent including an aryl group. Preferred quaternary ammonium compounds include lauryl benzyl ammonium chloride, cetyl trimethyl ammonium bromide, 1-aminoethyl-alkyl imidazoline, benzyl ammonium chloride (BAC), alkyl dimethyl benzyl ammonium chloride (AAC) and dodecyl dimethyl ammonium chloride (DDAC). Alternatively, a mixture of two or more quaternary ammonium compounds may be employed. A mixture of approximately equal amounts of AAC and DDAC has been found to be particularly advantageous. Unless otherwise stated, any reference to the amounts of quaternary ammonium compounds present in a composition refers to the total amount of all such compounds.

Preferably there is 0.001 to 1%, especially 0.01 to 0.1% of the quaternary ammonium compound or compounds present in the compositions of the present invention.

The sequestering agent, when present, serves to stabilise the peracetic acid generated by the oxygen donor and the peracid precursors. The sequestering agent further serves to enhance the fat- and protein-removing ability of the compositions of the present invention and interacts with the quaternary ammonium compound. This is not, however, an essential ingredient. This component when included is intended to sequester metal ions and the most suitable sequestering agent will be in part determined by the other components of the composition. Preferred sequestering agents are methylene phosphonic acids, for example diethylene triamine pentamethylene phosphonic acid, preferably the sodium salt thereof. EDTA is also acceptable but is less effective than the methylene phosphonic acids. Preferably there is 0.001 to 2% of the sequestering agent present, more preferably 0.01 to 1%, especially 0.1 to 0.5%.

The alcohol, when present, may be monohydric or polyhydric, with isopropyl alcohol being preferable. The preferred amount of alcohol, when present, is from 0.001 to 2%, more preferably from 0.01 to 0.5%.

An acidifier, when present, serve to reduce the pH of the formulation to a maximum of 9, but more preferably to a maximum of 8. Preferably the acidifier is an organic acid, especially a polycarboxylic acid. Examples of suitable acids include but are not restricted to: citric acid, EDTA, oxalic acid, phthalic acid, succinic acid, adipic acid, and lactic acid. A particularly preferred acid is citric acid.

Preferably the at least one acid has a pKa of between 1 and 5, more preferably, between 2 and 4, most preferably about 3.

The acidifier, when present, is preferably present in an amount of 0.001 to 2%, and more preferably 0.01 to 0.5%.

A base, when present, may be preset in an amount from 0.001 to 2%, more preferably 0.01 to 1%, most preferably 0.05 to 0.5%. Preferred bases include alkali metal carbonates or bicarbonates, especially sodium carbonate.

A corrosion inhibitor may also be optionally included. The corrosion inhibitor may, for example, be benzotriazole, tolytriazole, quaternary ammonium salts, for example quaternary ammonium alkylcarbonates, and polyacrylic acids.

If the formulation is a liquid formulation, the balance of the composition is de-ionised water with the balance of the composition optionally including an oxygen donor to enhance the cleaning properties of the composition.

Alcohol, colours and fragrances may also be optionally included in the liquid formulations.

If the formulation is a dry powder formulation, the balance of the composition is provided by a solid bulking agent which serves to absorb any water present in the non-ionic surfactant or other components thereby providing a dry composition.

The cationic surfactant or source of cations, when present, is intended merely to moderate the activities of the other components and as such represents an optional component.

Powder formulations are obtained by incorporating the active components in the powder. Sodium carbonate is a particularly preferred vehicle because it can be easily incorporated into a formulation, it provides an ideal base for incorporating the active chemicals and it also contributes towards water softening. There is also the advantage that the pH of the solution will remain above pH 9. Other suitable powders include magnesium sulphate, potassium carbonate, sesquicarbonate, sodium bicarbonate and borax. Optionally, an oxygen donor, colours and fragrances may be included in the balance of the solid composition.

The essential part of the formulation consists of a surfactant, a metal silicate or silicate complex (especially metasilicate) as an emulsifier, a phosphate compound as a water softener, and a quaternary ammonium compound as a complexing agent. The alcohol, when present, has the effect of improving the appearance of the solution by improving its clarity.

The exact relationship between the components is not clear but it is believed that they interact with one another in a synergistic manner. However, it is thought that the silicates have wetting, emulsifying and soil suspending properties and the polyphosphates function by sequestering hard water salts.

Silicates and silicate complexes also have a good buffering action, which means that in the presence of acidic soils, an alkaline pH is maintained almost until they have been exhausted. Some stainless steels are not totally “stainless”, and the presence of silicates or silicate complexes, coupled with the presence of phosphates or polyphosphates or orthophosphoric acid, will inhibit corrosion. Silicates also inhibit aluminium alloy corrosion, especially when phosphates or polyphosphates or orthophosphoric acid are present.

There also appears to be some improved surface activity when phosphates or polyphosphates are used in conjunction with non-ionic surfactants. A secondary function of the phosphates appears to be as a suspending agent which allows lifted biofilm to be rinsed away more easily,

The presence of peracetic acid, as a preferred compound generated in situ e.g. from an activator and the oxygen donor gives substantial anti-microbial and anti-viral activity. It is believed this activity delivers optimal biocidal activity and is highly effective against a wide range of organisms, including MRSA, Pseudomonas aeruginasa and Esherenichia coli. The oxygen donor is thought to provide oxygen at the liquid/polymer surface where its activity enhances biofilm removal from most plastic materials and in particular from nylon 66 and polypropylene.

Depending on the particular application intended for a composition useful in the method a number of additional components, can be incorporated within the formulations of the present invention. These include foam control agents, which preferably are silicones. Colours and fragrances may also be included as required. The percentage of these components in the composition are determined by end use requirements.

The method of the invention may involve the treatment of ductwork (for example plastics tubing), and if wished other apparatus used for water supply (for example water containers, caps, valves, flow restrictors etc) by immersion in the sanitizing composition. Alternatively, and preferably, it involves passing the sanitizing composition through the ductwork. Most preferably it may involve both measures, carried out in the following manner: the composition is run through the ductwork; and the flow is interrupted with sanitizing composition in the ductwork for a treatment interval. In this dual embodiment the sanitizing composition is then let out from the ductwork, and water is run through the ductwork to flush out the ductwork. The treatment interval is preferably at least 15 minutes, more preferably at least 30 minutes, and most preferably at least 2 hours. Suitably it may be up to 18 hours, preferably up to 6, most preferably up to 4 hours. It could be overnight, with the first operations being carried out when the last dental patient has been seen, and the subsequent operations being carried out before the first patient is seen, the next day.

Preferably the method of the invention is applied to sanitize small-bore ductwork, preferably in a dental facility. Preferably the lumen of the ductwork is less than 1.5 cm, more preferably less than 1 cm.

Preferably the pressure of water at the outlet of the apparatus is preferably not greater than 0.4 Atm (4×10⁴ Pa), more preferably not greater than 0.25 Atm (2.5×10⁴ Pa).

The following examples are illustrative of the invention.

### EXAMPLE 1

A solid state formulation was made by mixing the following solid state components:

| | |
|---|---|
| Sodium carbonate | 16% |
| Sodium metasilicate | 7% |
| Tetra sodium pyrophosphate | 22% |
| Non-ionic surfactant | 2% |
| Quaternary ammonium compound | 1% |
| Sodium perborate monohydrate | 30% |
| TAED | 15% |
| Citric acid | 5% |
| Diethylene triamine | |
| pentamethylene phosphonic acid | |
| sodium salt | 2% |

An aqueous solution of this formulation was prepared, for use in a method of sanitizing ductwork and fittings associated with the supply of potable water in a dental facility. This was done by dissolving 10 g of the composition in one litre of water. This solution was then used for cleaning dental ductwork and fitting to remove biofilm. The standard one litre canister of water used as potable water was replaced by a one litre canister of the treatment solution noted above. The composition was run in through the high speed turbines of the system, but at slow speed, through the sonic scaler and 3-in-1 water lumen, until approximately 0.75 litre had passed. The tap was closed and the apparatus left for 30 minutes. The water bottle receiver was then disconnected and flushed out with purified water then refilled with purified water and all water lines were flushed out again for 1 minute duration.

Fittings may be cleaned by over night immersion using the same solution.

With heavily contaminated apparatus the effectiveness is confirmed by the appearance of slimy deposits—biofilm and associated soiling—in the run-off liquid.

The aqueous solution of the invention showed highly effective sanitizing results.

### EXAMPLE 2

A concentrated liquid formulation was made by mixing the following components:

| | |
|---|---|
| Non-ionic surfactant | 8% |
| Sodium metasilicate | 5% |
| Tetra sodium pyrophosphate | 15% |
| Quaternary ammonium compound | 4% |
| Sodium carbonate | 15% |
| Propan-2-ol | 5% |
| De-ionised water | 48% |

This solution could be used either concentrated or in dilute form for sanitizing fittings associated with the supply of potable water in a dental facility. Typically, when used in diluted form, 1 part of this composition may be diluted with 10 to 100 parts water. Similar soil removal levels were obtained using this solution as were obtained with the formulation of Example 1.

### EXAMPLE 3

A following liquid formulation is used to sanitize dental apparatus, made from the following neat components, admixed:

| | |
|---|---|
| Sodium carbonate | 3% |
| Tetrapotassium pyrophosphate | 9% |
| Sodium metasilicate | 5% |
| Non-ionic surfactant | 2% |
| Quaternary ammonium compound | 1% |
| De-ionised water | to 100% |

To make a concentrate liquid the above solid composition is mixed into water at a ratio of 100 g of the solid composition to 1 litre of water. For use, the resulting concentrate liquid may be diluted further, typically at a ratio of 1 part by weight of concentrate liquid per 10 parts of water.

### EXAMPLE 4

A concentrated liquid formulation was made by mixing the following components:

| | |
|---|---|
| Non-ionic surfactant | 8% |
| Sodium Metasilicate | 5% |
| Tetra sodium pyrophosphate | 15% |
| Mixture of quaternary ammonium compounds | 8% |
| Sodium carbonate | 15% |
| Propan-2-ol | 5% |
| De-ionised water. | 44% |

In general the liquid formulations useful in the method of the present invention may be used either in concentrated form or at dilutions up to 100 times. Solid formulations may be diluted with water and are preferably diluted in the range 5 to 100 g of solid composition per litre of water, and more preferably in the range 10 g to 20 g of solid composition per litre on the combined grounds of efficiency and economy.

The compositions useful in the method of the invention are highly effective for sanitizing ductwork and fittings associated with the supply of potable water in a dental facility. When used for sanitizing ductwork, a composition containing an oxygen donor and preferably an activator therefor is preferred.

In this specification any explanations given as to how the invention works or of the function performed by individual components or of two or more components in combination represents the beliefs or expectations of the inventor(s) and is not to be regarded as limiting the invention. Ultimately the key point about the invention is that it is highly effective, however that may happen.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A method of sanitizing ductwork associated with the supply of a potable liquid, the method comprising applying to the ductwork an aqueous sanitizing composition comprising:
a metal silicate or silicate complex;
a metal phosphate or polyphosphate;
or orthophosphoric acid; and
a non-ionic or amphoteric surfactant.

2. A method as claimed in claim 1, wherein the metal cation in the silicate or silicate complex is an alkali metal cation or an ammonium cation.

3. A method as claimed in claim 1, wherein the metal phosphate or polyphosphate is an alkali metal or ammonium phosphate and the phosphate is mono-, di- or tribasic.

4. A method as claimed in claim 1, wherein
the surfactant is nonyl phenol ethoxylate.

5. A method as claimed in claim 1, wherein there is present a quaternary ammonium salt, preferably derived from a lower alkyl amine having one or more alkyl substituents each being C6 or less and a single substituent including an aryl group.

6. A method as claimed in claim 1, comprising an oxygen donor.

7. A method as claimed in claim 6, comprising an activator of the oxygen donor.

8. A method as claimed in claim 6, wherein the composition further comprises a sequestering agent.

9. A method as claimed in claim 1, wherein the potable liquid is supplied under low pressure from a container of potable liquid.

10. A method as claimed in claim 1, wherein the potable liquid is water.

11. A method as claimed in claim 1, being a method for sanitizing ductwork and associated with the supply of potable liquids in a dental treatment facility.

12. A method as claimed in claim 1, the method further comprising the step of: producing the sanitizing composition by diluting a concentrate composition with water, before applying the sanitizing composition to the ductwork; wherein when the composition is in liquid form the dilution is in an amount of between 10 ml and 100 ml of the liquid concentrate composition per litre of water; and wherein when the concentrate composition is in solid form and the dilution is in an amount of between 5 g and 100 g of the solid concentrate composition per litre of water.

13. A method as claimed in claim 1, wherein the sanitizing composition is run through the ductwork; the flow is interrupted, with sanitizing composition in the ductwork, for a treatment interval; the sanitizing composition is then let out from the ductwork; and water is run through the ductwork to flush out the ductwork.

14. A method as claimed in claim 1, wherein the ductwork is in a dental facility, the ductwork being part of a water supply system in which water is fed to a patient at a water pressure, at the outlet of the apparatus, of greater than 0.4 Atm (4×10⁴ Pa) through small-bore piping having a lumen of less than 1.5 cm.
